(19) United States
(12) Patent Application Publication
(10) Pub. No.: US 2026/0084195 A1
(43) Pub. Date: Mar. 26, 2026

(54) A WASTE PROCESSING APPARATUS, AND A METHOD OF PROCESSING WASTE

(71) Applicant: Sangenic International Limited, Newcastle Upon Tyne (GB)

(72) Inventor: Joshua Hume, New Castle Upon Tyne (GB)

(21) Appl. No.: 19/111,524

(22) PCT Filed: Sep. 14, 2023
(86) PCT No.: PCT/GB2023/052385

§ 371 Date: Mar. 13, 2025

(30) Foreign Application Priority Data
Sep. 14, 2022 (GB) 2213482.9

(51) Int. Cl. B09B 3/40; B02C 19/18; B09B 3/35; B09B 3/38; B09B 101/25; B09B 101/67; B09B 101/70

(52) U.S. Cl. CPC B09B 3/40; B02C 19/18; B09B 3/35; B09B 3/38; B09B 2101/25; B09B 2101/67; B09B 2101/70

(57) ABSTRACT

The present invention relates to a waste processing apparatus, particularly an apparatus for processing used nappies. The waste processing apparatus includes a chamber including a first opening through which a portion of waste is received into the chamber; and a heating member adapted to provide heat to the portion of waste in the chamber. A method of processing waste, particularly a method of processing used nappies, is also disclosed.

The present invention relates to a waste processing apparatus, particularly an apparatus for processing used nappies. A method of processing waste, particularly a method of processing used nappies, is also disclosed.

### INTRODUCTION

The problem of how to effectively store waste, particularly domestic waste such as waste foodstuff or disposable nappies, is well recognised. As used herein, the term ‘nappy’ includes items known as diapers such as the disposable diapers used with infants, as well as similar personal hygiene products such as used by older children and adults.

It is possible to temporarily store waste in ordinary household bins prior to disposal with household waste. Waste items, particularly used nappies, typically pack inefficiently when stored, meaning that the waste, such as the waste associated with a several nappy changes occupies a significantly large proportion of the volume of household waste.

Storage of odorous waste, such as nappies, in household bins is not sufficiently smell proof to enable the waste to be hygienically stored except on a very temporary basis. Plastic bags may be used to wrap used nappies and other waste in an attempt to block smell leakage, however these bags have limited effect and often comprise more plastic than is needed to wrap waste, such as one bag for each nappy, therefore creating unnecessary waste.

Beyond simply sending waste to landfill with household waste, it is possible to compost certain waste foodstuffs. However, the large variability of starting material and treatment conditions of the waste foodstuff means results are variable.

Once disposed in household waste, nappies are typically sent to landfill. Such nappies are expected to take many decades, if not longer, to fully decompose. The decomposition process will be further lengthened when the nappies have been wrapped in plastic for temporary storage. Furthermore, disposal of nappies overall requires transport and landfill storage of a very large numbers of used nappies, numbering perhaps millions daily. Such large numbers occupy a significant volume of landfill.

Certain known disposable nappies are composed of constituent materials which attempt to improve rates of decomposition of the nappy. Other waste may be susceptible to decomposition under certain conditions. Nevertheless, such decomposition will rely on natural decomposition processes, which are slow and easily inhibited by, for example the soiled contents of the nappies, or by associated waste materials and plastic wrapping. Furthermore, nappies still require the transport of very large volumes and masses of nappies to landfill, prior to decomposition.

Accordingly, it would be useful to solve, mitigate or obviate, at least partly, at least one of the problems and/or disadvantages associated with the prior art.

It would be able to satisfy the requirements associated with built-in household furniture, such as the most typical size, aesthetic and hygiene requirements. It would also be useful to provide waste disposal in a simple and inexpensive to produce apparatus that is operationally reliable.

### SUMMARY OF THE INVENTION

The invention is set out in the appended claims.

According to an aspect of the invention, there is provided a waste processing apparatus comprising:
a chamber including a first opening through which at least one portion of waste is received into the chamber; and
a heating member adapted to provide heat to the portion of waste in the chamber.

By applying heat to a portion of waste, for example a nappy, the apparatus dehydrates the waste, such as the nappy and its contents, thereby reducing its weight. Applying heat also disinfects the waste, such as the nappy and its contents, thereby reducing the odour produced during storage. In particular, the temperature may be sufficiently high to ensure bacteria are effectively killed.

Suitably, the apparatus may be a domestic waste processing apparatus. More suitably, the apparatus may be a nappy waste processing apparatus, or foodstuff processing apparatus. In a domestic environment there exists spatial and aesthetic requirements which are not considered to be important in industrial waste processing. In this way, the apparatus may be adapted for processing waste to minimise the occupied space and not negatively affect the design thereof. Moreover, the need for hygiene is greater in the domestic environment of the user than for industrial waste processing, so as prevent contamination of the living environment.

Suitably, a portion of waste may one or more of: a nappy or a portion of foodstuff waste.

Suitably, the apparatus may be dimensioned to fit on a worksurface, or within an enclosure or furniture of a kitchen, such as a worksurface or cupboard in a kitchen, or a domestic worktop. For example, the apparatus may be have one or more of a height, a width, or a depth of less than 1 m.

Suitably, the apparatus may be freestanding.

Suitably, the apparatus may be configured to operate using a domestic electrical power supply. The domestic power may be AC current supplied with a voltage in nominal range of from 100 volts to 400 volts, more suitably in a nominal range of from 120 volts to 240 volts. The domestic power may be supplied with a voltage at a nominal 100 volts, 200 volts, 220 volts, 230 volts, 240 volts, 380 volts or 400 volts.

Suitably, the apparatus may have an electrical power consumption, typically a peak electrical power consumption of: 4 kilowatts or less, 3 kilowatts or less, or 2 kilowatts or less.

Features of a waste disposal apparatus disclosed herein may be features of either of a nappy processing apparatus or a waste food processing apparatus according to an aspect of the invention.

Suitably, the chamber may be rotatable.

Suitably, the chamber may be a container including a side wall extending from a base to an upper portion. The upper portion may include the first opening and, optionally, a lid to close or partially close the opening.

Suitably, the chamber may include a central longitudinal axis. In certain examples, the chamber is rotatable about its central longitudinal axis.

Suitably, the chamber may be removably mounted in the apparatus.

Suitably, the chamber may include a base and a side wall extending away from the base. More suitably, the side wall may be tapered or conical. In this way, the chamber may be narrower at the base than distal to the base.

Suitably, the base wall and/or the side wall may be metallic. In this way, the base wall and/or side wall may be thermally conductive so that heat from the heating member is spread around the chamber. The waste in the chamber is thereby heated more evenly.

In certain examples, the apparatus is configured for batch processing of waste. In this way, a user may deposit waste, for example several nappies or several portions of food waste, into the chamber for processing concurrently.

Suitably, the chamber may be adapted to receive at least five nappies, preferably five to ten nappies, of at least size 5.

Suitably, the chamber may have a capacity of at least 2 litres, preferably 2 to 5 litres. The dehydration of the waste within the chamber significantly reduces the volume of the waste. The processed waste requires a greatly reduced volume for subsequent storage or disposal. For example, if the waste is nappy waste then the volume may be reduced by up to 90% once processed. Any downstream receptacle for the processed waste, such as a receptacle associated with the apparatus thereby has an effective storage capacity for an amount of waste of considerably greater volume before it was processed by the apparatus.

Suitably, the chamber may form a passageway for processing waste as it passes through the chamber. In this way, the chamber may be akin to an oven, with a first opening through which at least one portion of waste is received into the chamber and heated by the heating member.

Suitably, the heating member may be a radiating heating member or a convection heating member. More suitably, the heating member may be a halogen lamp mounted within the chamber.

Suitably, the chamber may include a second opening configured to discharge the processed waste from the chamber. The user may deposit waste into the first opening which is then processed as it passes through the chamber to the second opening. Thus, the apparatus may be configured for continuous processing of waste. In this way, the user can deposit a first portion of waste, such as a first nappy, into the apparatus for processing, and then deposit a second portion of waste, such as a second nappy, at any time interval after the first portion.

Suitably, the base wall may include the second opening. More suitably, the second opening is a series of apertures extending through the base wall. Typically, the series of apertures are several centimetres wide, for example in a range of from 2 centimetres to 15 centimetres wide, preferably in a range of from 8 centimetres to 12 centimetres wide, more preferably with a width of substantially 10 centimetres. In this way, the openings are sized to retain the waste within the chamber until the waste is sufficiently processed to form small fragments. Typically, when the second opening is provided in the base wall, waste passes from top to bottom of the apparatus during processing. The waste is deposited into the first opening at the top of the apparatus and is discharged from the second opening at or near the foot of the apparatus.

Suitably, the apparatus may include a conveyor assembly to convey waste from the first opening to the second opening. The conveyor assembly may include a suitable moveable member, typically driven by an electric motor, to convey waste from the first opening to the second opening. The moveable portion may include one or more of: a moveable belt, a roller, a container, a rotatable screw, a slat, or a surface portion. The moveable portion may be carried by a moveable support member, typically a moveable support member driven by an electric motor.

Suitably, the conveyor assembly may include at least one moveable belt, preferably at least two moveable belts, extending from the first opening to the second opening of the chamber. More suitably, the moveable belt or moveable belts extend beyond the chamber. That is, the moveable belt or belts extends upstream of the first opening and/or or extends downstream of the second opening.

Typically, when a conveyor assembly is used to convey waste from the first opening to the second opening, the waste passes from laterally through the apparatus during processing. In certain examples, the conveyor assembly may be inclined so that the waste moves laterally through the apparatus and is discharged from the apparatus at a lower point to where it is deposited.

Suitably, the conveyor assembly may include a pair of moveable belts oriented to cooperatingly engage the waste and convey the waste through the chamber. More suitably, the conveyor assembly is operably connected to a controller. The controller may also be connected to a heating member as described herein. The controller may be configured to adjust a speed of the conveyor assembly to determine the duration that the waste is within the chamber during processing. That is, the controller may control a dwell time of the waste in the chamber by adjusting the speed of the conveyor assembly. Additionally, or alternatively, the controller may be configured to determine the temperature to which the waste in the chamber is heated. The controller may be configured to enable the heating member to provide continuous heat to the waste in the chamber or intermittent heat to the waste in the chamber.

Suitably, a first belt of a pair of moveable belts may be oriented to move along a first axis and a second belt of the pair is oriented to move along a second axis. More suitably, the first axis and the second axis are non-parallel to one another. The first axis and the second axis may be oriented so that the first belt and the second belt move towards one another as they approach the second opening. In this way, the moveable belts apply in an increasing compression force to the waste as it is conveyed towards the second opening.

Suitably, the apparatus may also include a lid for selectively closing the first opening. More suitably, the lid may form an air-tight seal with the first opening in use. The lid may be configured to be releasably mounted to the first opening. The lid may be mounted to the first opening by any suitable means, for example via a screw thread or bayonet fixing arranged to engage with a corresponding feature on the apparatus. Suitably, the lid may include an inlet through which a portion of waste is received into the first opening of the chamber. The inlet may include a moveable closure means, such as a plunger or cover, configured to selectively seal the inlet, for example when the apparatus is in use.

Suitably, the moveable closure means may include a roller arranged in the inlet. The roller has a horizontal rotational axis. The roller may include an outer surface with a recessed channel in the outer surface. The recessed channel extends along the outer surface in an axial direction. In this way, in use, after depositing a portion of waste into the inlet, the user actuates the roller which rotates the roller so that the recessed channel captures the waste and pulls it through the inlet. In certain examples, the moveable closure means may include a pair of rollers arranged in the inlet with parallel rotational axes. In this way, the user actuates both rollers to rotate in opposing angular directions so that channels in the respective roller outer surfaces cooperatingly capture the waste and pull it through the inlet.

Suitably, the roller, or pair of rollers, may be actuated manually, for example by moving a handle, or by activating an electric motor to drive the roller or rollers.

Suitably, the axes of the pair of rollers may move relative one another to allow the rollers to move apart when the waste is pulled through. The rollers may be made from a rigid material, or may be elastic to better grip the waste.

Suitably, the apparatus may include at least one agitating member. More suitably, the agitating member includes one or both of least one blade member or at least one contacting element.

at least one blade member arranged to project into the chamber, wherein the at least one blade member is actuatable so as to agitate the waste, such as at least one nappy, in the chamber.

Suitably, the at least one blade member is rotationally mounted within the chamber, typically to rotate around the central longitudinal axis of the chamber. More suitably, the at least one blade member and the heating member are rotationally mounted within the chamber to co-rotate around the central longitudinal axis of the chamber. In this way, the apparatus may be configured so that the a single electric motor may drive the rotational movement of both the blade member and the heating member.

Suitably, the at least one blade member may project into the chamber from a wall portion of the chamber. Suitably, the at least one blade member may project into the chamber from the heating member.

Suitably, the apparatus may include at least one contacting element, wherein each contacting element is adapted to at least partially disintegrate the waste. More suitably, the contacting element may include a roughened surface, a plurality of projections, teeth or serrations, or may include one or more patterned or textured regions. The plurality of projections, plurality of teeth, plurality of serrations, patterned region or textured region may be formed of either a regular arrangement, or an irregular arrangement, of a number of individual components.

In this way, the contacting element pulls at outer portion of the waste, such as the surface layers of the nappy, to initiate disintegration. Further processing of the waste may cause the contacting element to abrade or cut through the waste, for example the fibres of the nappy, accelerating disintegration. Consequently, the associated heat dehydrates the waste more quickly and evenly. Consequently, odours within the waste are deodorised more quickly due to exposure to air.

Suitably, the at least one contacting element may be mounted on a blade member.

Suitably, the contacting element may be mounted to or formed on any surface of the apparatus to cause disintegration of the waste by relative movement between the waste and the contacting element. For example, the contacting element may be mounted to, or formed on one of: a chamber, a blade member, a shredding assembly or a conveyor assembly. The contacting element may form part of one or more of: a roller, a drum, a barrel, or a moveable belt.

Suitably, the apparatus may include a shredding assembly. The shredding assembly includes at least one contacting element moveably mounted to the shredding assembly, preferably rotatably mounted therein. The shredding assembly may be positioned at an entrance to the first opening, typically arranged over the entrance to the first opening. In this way, the shredding assembly is upstream of the chamber so that the waste is substantially disintegrated prior to heating in the chamber.

Suitably, the shredding assembly may include a rotating barrel with the at least one contacting element provided on the barrel. For example, the contacting element may be a plurality of projections, teeth or serrations provided on an outer surface of the rotating barrel. The rotating barrel may be arranged in the shredding assembly with a horizontal rotational axis. In this way, when the user actuates the shredding assembly, the roller rotates so that the projections, teeth, or serrations disintegrate the waste. When the waste is disintegrated into sufficiently small fragments, the barrel conveys the fragments into the chamber.

Suitably, the shredding assembly may include a pair of rotating barrels, each including a respective contacting element. Each contacting element may be a plurality of projections, teeth or serrations provided on an outer surface of the rotating barrel. Preferably, the projections, teeth or serrations of each barrel are interlaced with one another. The pair of rotating barrels may be arranged in the inlet with parallel rotational axes. In this way, when the user actuates the shredding assembly, both barrels rotate in opposing angular directions so that the respective projections, teeth, or serrations of each barrel cooperatingly capture and disintegrate the waste, thereby increasing the speed with which waste is disintegrated. When the waste is disintegrated into sufficiently small fragments, the barrels convey the fragments into the chamber.

By arranging a shredding assembly upstream of the chamber, the waste may be substantially disintegrated prior to heating. The waste is thereby processed more effectively by heat treatment within the chamber. The associated heat dehydrates the waste more quickly and evenly, and odours within the waste are deodorised more quickly due to exposure to air. The overall waste processing is more efficient because heating time is substantially reduced.

Suitably, the shredding assembly may be configured to perform a cleaning cycle. Most suitably, the cleaning cycle includes counter-rotating the barrel or barrels. By reversing the direction of rotation of the barrels from the processing direction, any residue or particulates that may be trapped in the contact element or elements are more readily dislodged.

Suitably, the heating member may be a projection extending into the chamber, typically from the base of the chamber. The heating member may project along the central axis of the chamber. Suitably, a proximal portion of the heating member is mounted to the base and a distal portion of the heating member is disposed away from the base. The proximal portion may be a proximal end portion of the heating member. The distal portion may be a distal end portion of the heating member.

Suitably, the proximal portion and the distal portion are of substantially the same width. Alternatively, the proximal portion may be wider than the distal portion, for example so that at least a section of the heating member is a conical shape. Alternatively, the proximal portion may be narrower than the distal portion, for example so that at least a section of the heating member is an inverted conical shape.

Suitably, the at least one contacting element may be mounted on the heating member projecting into the chamber. More suitably, the at least one contacting element may be an elongate rib extending around the heating member, for example, an elongate helical rib extending around an outer surface of the heating member. The helical rib may extend around the heating member from the proximal portion to the distal portion of the heating member. Suitably, the at least one contacting element may be mounted on the side wall projecting into the chamber. More suitably, the at least one contacting element may be an elongate rib extending around the side wall, for example, an elongate helical rib extending around an inner surface of the side wall. The helical rib may extend around the side wall in away from the base.

Suitably, the side wall with the elongate helical rib, is conical so that narrower at the base than distal to the base. In this way, the space between the side wall and the heating member decreases towards the base so that, as the waste is disintegrated into smaller fragments, these fragments move towards the base. The fragments of waste are thus distributed along the axial height of the chamber, exposing a larger proportion directly to the contacting element and/or the heating member. The speed of processing the waste is thereby increased.

Suitably, the at least one blade member may include a first blade member and a second blade member, wherein the vertical height in the chamber of the first blade member is different to the vertical height in the chamber of the second blade member.

Suitably, the heating member may provide heat to increase a temperature of the at least one portion of waste to, for instance at least 50° C., for instance at least 60° C., for instance at least 70° C. Suitably, the heating member may provide heat to increase a temperature of the at least one portion of waste in a range, for instance from 50° C. to 90° C., for instance from 60° C. to 90°, for instance 70° C. to 90° C., for instance from 50° C. to 80° C., for instance from 60° C. to 80°, for instance 70° C. to 80° C., for instance from 50° C. to 70° C., for instance from 60° C. to 70°, for instance 50° C. to 60° C. Suitably, the temperature may be selected according to the type of waste. In this way, the temperature may adjusted to ensure any bacteria in the waste are effectively killed.

Suitably, the heating member may be adapted to provide heat for a predetermined period of time. The period of time may be, for example, 3 hours, 4 hours, 5 hours, 6 hours, 7 hours, 8 hours or more. The period of time may be adjusted depending on the temperature to which the waste is heated, such that the duration of heat is adjusted according to the overall cycle time required by the user. A required cycle time of 10 hours may mean the temperature is raised to at least 50° C., typically 50-60° C., for at least 8 hours. A required cycle time of 4 hours may mean the temperature is raised to at least 70° C., typically 70-80° C., for at least 3 hours. The remaining time in any cycle may be employed to allow the processed waste to cool. Suitably, the duration of the heating, or the cooling part of the cycle may be selected according to the type of waste, so that dehydration and/or disinfection of the waste is effective and efficient.

Suitably, the heating member may be adapted to provide heat intermittently.

Suitably, the heating member may be arranged so that at least a portion of the heating member projects into the chamber. More suitably, at least a portion of the heating member may project from the base of the chamber.

Suitably, the heating member may include an electrical heating element. More suitably, the electrical heating element may be operably connected to a controller.

Suitably, the apparatus may include a rotator. More suitably, the rotator may be a rotating arm or member to operably engage and rotate the chamber. Alternatively, the rotator may be an axle or spindle to operably engage rotate one or both of: the heating member and the blade member.

Suitably, the apparatus may also include a conduit for extracting air from chamber. In this way, a negative pressure relative to the ambient atmosphere may be maintained within the chamber during use. Odours from the waste or odours generated during processing are thereby held within the chamber instead of escaping into the surrounding environment. Typically, the conduit includes a fan assembly to draw air from the chamber into the chamber.

Suitably, the conduit may be fluidly connected to a filter element. More suitably, the filter element may be configured to deodorise, or to trap or collect gas or vapour from the air passing through the conduit from the chamber. Yet more suitably, the filter element may be configured to selectively remove certain chemicals from the air passing through the conduit from the chamber. For example the filter element may include a deodorise material to remove odours from the air, or an absorber for certain groups of chemicals, before it is discharged into the ambient air. The filter element may include activated carbon.

Suitably, the apparatus may also include a storage chamber for holding processed waste.

Suitably, the apparatus may also include an ultraviolet lamp configured to irradiate the waste during processing. In this way, the waste may be more effectively sterilised during processing.

Suitably, the apparatus may include a detector configured to detect the insertion of waste into the first opening.

Suitably, the apparatus may include a detector configured to detect the insertion of waste into the second opening.

Suitably, the apparatus may include a detector configured to detect the insertion of waste into the chamber.

Suitably, the detector may be operably connected to a controller to provide a signal to the controller to indicate that waste has been inserted into the first opening, the second opening or the chamber. The controller may be configured, in response to a signal received from the detector, to activate one or more of: the contacting member, the heating member, the lid, the moveable closure means, the shredding assembly, the ultraviolet lamp, the fan assembly or the conveyor assembly. In this way, the apparatus may provide automated processing of waste, further improving the efficiency of the device.

According to an aspect of the invention, there is provided a waste processing apparatus comprising:
a chamber including a first opening through which at least one portion of waste is received into the chamber; and
an agitating member adapted to agitate to the portion of waste in the chamber.

Suitably, the agitating member may be a blade member or a contacting element.

According to another aspect of the invention, there is provided a method of processing a waste, the method including:
depositing at least one portion of waste into a chamber through a first opening of the chamber; and
providing heat to the at least one portion of waste within the chamber.

Suitably, the method is a method of processing a nappy.

Suitably, the method may also include a step of cooling the processed waste.

Suitably, the step of providing heat may increase a temperature of the at least one waste to for instance at least 50° C., for instance at least 60° C., for instance at least 70° C. Suitably, the step of providing heat may increase a temperature of the at least one portion of waste to within a range of, for instance from 50° C. to 90° C., for instance from 60° C. to 90°, for instance 70° C. to 90° C., for instance from 50° C. to 80° C., for instance from 60° C. to 80°, for instance 70° C. to 80° C., for instance from 50° C. to 70° C., for instance from 60° C. to 70°, for instance 50° C. to 60° C. Suitably, the step of providing heat may increase a temperature of the at least one portion of waste to, for instance at least 50° C., or for instance at least 70° C.

Suitably, the step of depositing the at least one portion of waste into the chamber may include using a conveyor assembly to convey the waste from the first opening into the chamber.

Suitably, the method also includes conveying the waste from the chamber to the second opening so discharge the processed from the second opening.

Suitably, the conveyor assembly may include a moveable member, typically driven by an electric motor, to convey waste from the first opening to the second opening. The moveable portion may include one or more of: a moveable belt, a roller, a container, a rotatable screw, a slat, or a surface portion. The moveable portion may be carried by a moveable support member, typically a moveable support member driven by an electric motor.

The conveyor assembly may include a pair of moveable belts oriented to cooperatingly engage the waste and convey the waste through the chamber. The pair of moveable belts may be configured to apply a compressing force to the waste. Typically, the pair of moveable belts apply an increasing compressing force to the waste as the belts approach the second opening.

Suitably, the method may also include a step of agitating the at least one portion of waste within the chamber. The step of agitating the waste may occur one or more of: before the step of providing heat to the waste, after the step of providing heat to the waste, or concurrently with the step of providing heat to the waste.

Suitably, the method may also include agitating the at least one portion of waste prior to depositing the at least one portion of waste into the chamber. More suitably, the step of agitating the at least portion of waste may include using a shredding assembly as described herein. By arranging a shredding assembly upstream of the chamber, the waste may be substantially disintegrated prior to heating.

In these ways, the steps of providing heat and agitating the waste may be varied according to the type of waste. The order of the steps may be adjusted to ensure different types of waste are processed in the most effective way. The combination of agitation and associated heat dehydrates the waste more quickly and evenly. Odours within the waste are deodorised more quickly due to exposure to air. The overall processing of waste is more efficient because heating time is substantially reduced.

Suitably, the step of agitating the at least one portion of waste within the chamber may include actuating a blade member arranged to project into the chamber.

Suitably, the step of agitating the least one portion of waste may include partially disintegrating the waste with a contacting element. More suitably, the step of agitating the waste may include using a shredding assembly as described herein.

According to another aspect of the invention, there is provided a method of processing waste, the method including:
depositing at least one portion of waste into a chamber through a first opening of the chamber; and
agitating the at least one portion of waste within the chamber.

Suitably, the method may also include the step of providing heat to the at least one portion of waste within the chamber. The step of providing heat to the waste may occur one or more of: before the step of agitating the waste, after the step of agitating the waste, or concurrently with the step of agitating the waste.

As will be apparent, features, integers, characteristics or groups described in conjunction with a particular aspect, embodiment or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

As used herein, agitation means to move, grind, apply pressure to or otherwise cause waste, such as foodstuff, or nappies held in the chamber to be exposed more effectively to air in the chamber. Agitation may be accompanied by an additional action, for example a physical action such as cutting or abrasion. A physical action during processing, such as may be provided by a contacting member described herein, may cause disintegration of the waste.

Certain examples accelerate the rate of dehydration of the waste. In particular, certain examples accelerate the ageing of nappies. That is nappies, particularly disposable nappies, are processed after use in a manner which would speed up decomposition of the nappies. Certain examples ensure decomposition of the waste may commence even during temporary storage.

Certain examples reduce the smells produced when storing waste after use, particularly during temporary storage of nappies employed by parents and carers.

Certain examples process waste in an energy efficient manner. Waste, particularly disposable nappies, are processed in a manner which reduces their weight when stored after use within household or commercial waste. Certain examples process the waste so as to reduce its weight by dehydrating the waste and its contents. In particular, certain examples ensure heat is applied evenly to the waste, increasing efficiency and reducing processing times.

Certain examples result in waste, particularly disposable nappies, processed after use in a manner which increases the efficiency of packing when stored after use within household or commercial waste. The dehydration of the waste within the chamber significantly reduces the volume of the waste and disintegration aids packing efficiency. The processed waste requires a greatly reduced volume for subsequent storage or disposal. For example, if the waste is nappy waste then the storage volume may be reduced by up to 90% once processed. Any downstream receptacle for the processed waste, such as a receptacle associated with the apparatus thereby has an effective storage capacity for an amount of waste of considerably greater volume before it was processed by the apparatus.

Certain examples process the waste into fragments, that is the examples causes waste to disintegrate during processing. The fragments of waste are in a form that is more easily handled. The fragments can be more efficiently packed within household waste or in landfill because fragments can be packed into a reduced volume. Furthermore, fragmented waste in a form which is more readily decomposed because an increased proportion of the waste is exposed to its environment. Fragmented and dried waste may be more readily decomposed because the starting material has a more consistent water content from batch to batch.

### BRIEF DESCRIPTION OF THE DRAWINGS

Embodiments of the invention are now described, by way of example only, hereinafter with reference to the accompanying drawings, in which:

FIG. 1 shows a cross-sectional schematic view of a first example nappy processing apparatus;

FIG. 2 shows a cross-sectional schematic view of a second example nappy processing apparatus;

FIG. 3 shows a first example method of processing a nappy;

FIG. 4 shows a second example method of processing a nappy;

FIG. 5 shows a cross-sectional view of the example of FIG. 2 with a nappy a) before, and b) after processing;

FIG. 6 shows an illustration of an example blade member;

FIG. 7 shows a cross-sectional schematic view of a third example nappy processing apparatus;

FIG. 8 shows a cross-sectional schematic view of an example waste processing apparatus;

FIG. 9 shows a cross-sectional schematic view of a second example nappy processing apparatus;

FIG. 10 shows a cross-sectional schematic view of a third example waste processing apparatus;

FIG. 11 shows a series of cross-sectional views of the moveable closure means of the example of FIG. 10;

FIG. 12 shows a cross-sectional schematic view of an example of a continuous waste processing apparatus; and

FIG. 13 shows a cross-sectional schematic view of another example of a continuous waste processing apparatus.

In the drawings, like reference numerals refer to like parts.

### Description of Examples

Certain terminology is used in the following description for convenience only and is not limiting. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, as used herein, the terms ‘connected’, and ‘mounted’ are intended to include direct connections between two members without any other members interposed therebetween, as well as, indirect connections between members in which one or more other members are interposed therebetween. The terminology includes the words specifically mentioned above, derivatives thereof, and words of similar import. Further, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

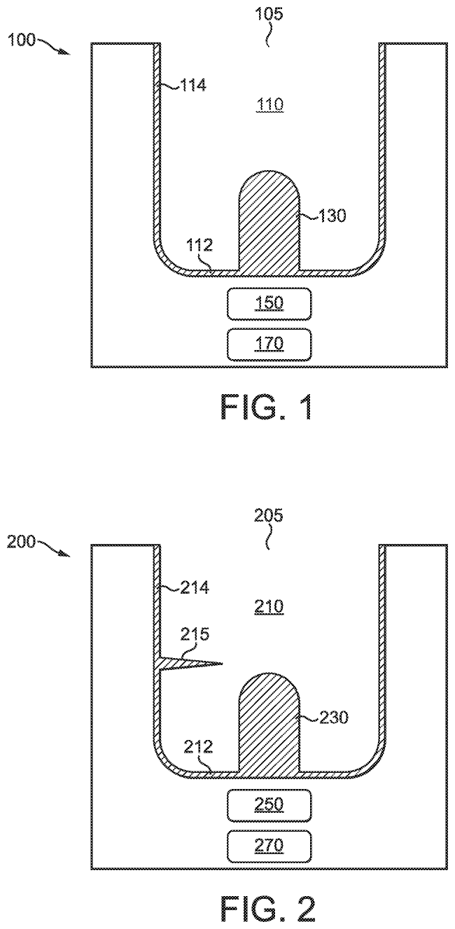

Referring now to FIG. 1, there is shown a nappy processing apparatus 100 including a chamber 110 with an opening 105 through which at least one nappy is received into the chamber 110. A heating member 130 is also provided, which is adapted to provide heat to the nappy in the chamber 110. As will be understood, while the apparatus 100 is described as a nappy processing apparatus, it would also be suitable for processing alternative domestic waste, such as waste foodstuffs.

The chamber 110 includes a base 112 and a side wall 114 extending away from the base 112. In the example shown, the side wall 114 extends from a closed end, including the base 112, to an open end including the opening 105. Together, the base 112 and side wall 114 enclose the chamber 110.

The chamber 110 is cylindrical, with a central axis extending normal to the base 112. The side wall 114 extends away from the base 112 in an axial direction. That is, the side wall 114 extends in a direction parallel to the central axis.

In the example shown, the opening 105 is an annular opening. The opening 105 is provided with a removable lid (not shown) configured to the fit within the opening 105. The removable lid selectively closes the opening 105, providing an airtight seal to the chamber 110.

The nappy processing apparatus includes a heating member 130 extending into the chamber 110 from the base 112. The heating member 130 projects along the central axis of the chamber 110. Thus, the heating member 130 extends into the chamber 110 by projecting axially from the base 112.

The heating member 130 includes an electrical heating element therein. The heating element is connected to a suitable electrical circuit, including a controller. The controller enables the heating element to be selectively activated, to control how the heating member 130 provides heat to the nappies in the chamber. Further examples and details about the operation of the heating member 130 are provided herein.

A proximal end of the heating member 130 is provided on the base 112. A distal end of the heating member 130 is disposed away from the base 112 and towards the centre of the chamber 110. The heating member 130 thereby has an increased surface area available to provide heat to the nappies. In this way, the heating member 130 is arranged to efficiently and effectively heat nappies received in the chamber 110.

The nappy processing apparatus 100 includes a rotator 150. The rotator 150 is mounted below the chamber 110 and operably engaged with the chamber 110 to provide rotation thereto. In an example, the rotator may be a rotating arm or member to operably engage and rotate the chamber. Alternatively, the rotator may be a spindle to operably engage one or both of a heating member or a blade member.

In the example shown, the chamber 110 is rotatably mounted in the apparatus 100. The wall 114, base 112 and the heating member 130 are integrally formed so as to be rotated together by the rotator 150. In alternative examples the chamber may be configured so that rotator provides rotation to one or more of the wall, the base, and the heating member. The rotator thereby may provide relative rotation between one or more of the wall, the base, and the heating member.

An electric motor 170 is provided to actuate the rotator 150. The rotator 150 includes suitable means (not shown) to provide rotational movement to the chamber 110, for example using a suitable belt drive.

The electric motor 170 is also connected to the controller. The controller thereby enables the motor to be selectively activated so as to selectively rotate the chamber 110 via the rotator. The controller may be configured to determine the rotational speed and the time period that the chamber 110 is rotated.

In the example shown, the controller is thereby configured to control both heating of the nappies in the chamber 110 as well as rotation of the chamber 110 itself. Efficient processing of the nappies is thereby provided. In further examples, a controller may be configured to control either the heating or the rotation of a component of the apparatus, such as the chamber or the heating member. Alternatively, heating and rotation may be directly controlled by the user, for example by actuating a switch to commence heating or a switch to commence rotation.

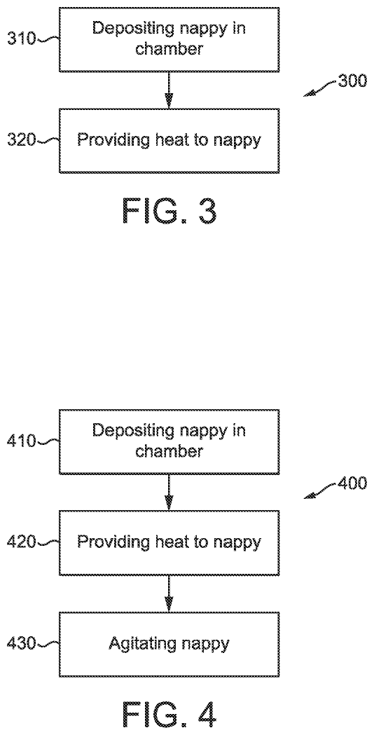

Referring additionally to FIG. 3, there is shown a flow chart illustrating a method 300 of processing a nappy. The method may be carried out using an example apparatus such as example apparatus 100 described with reference to FIG. 1. As will be understood, while the method 300 is described as suitable for processing a nappy, it would also be a suitable method for processing alternative domestic waste, such as waste foodstuffs.

A first step 310 includes depositing at least one nappy into a chamber through a first opening of the chamber. Suitably, the chamber includes a base and wall extending away from the base.

A second step 320 includes providing heat to the at least one nappy within the chamber. Suitably, the heat is provided by a heating member. The heating member may include, for example, a heating element and controller as described herein to selectively provide heat. Suitably the second step is initiated by a user, for example by actuating a switch, once the desired number of nappies have been deposited into the chamber.

Optionally, the second step 320 raises the temperature of the at least one nappy to at least 50° C. The temperature may be maintained for a time period sufficient to dehydrate the nappy. The time period may be, for example, 3 hours, 4 hours, 5 hours, 6 hours, 7 hours, 8 hours or more.

The time period may be adjusted depending on the temperature to which the nappy is heated, such that the method is adjusted according to the overall cycle time required by the user. A required cycle time of 10 hours may mean the temperature is raised to at least 50° C., typically 50-60° C., for at least 8 hours. A required cycle time of 4 hours may mean the temperature is raised to at least 70° C., typically 70-80° C., for at least 3 hours. The remaining time in any cycle may be employed to allow the processed nappy to cool.

As will be understood, in a method described herein but not shown in the figures, a method of processing waste is provided in which, in a first step, at least one portion of waste is deposited into a chamber through a first opening of the chamber. And, in a second the at least one portion of waste is agitated within the chamber. Optionally, a third step may provide heat to the waste within the chamber. Suitably, the heat is provided by a heating member.

Referring now to FIG. 2, there is shown another nappy processing apparatus 200. Where the features are the same as the example of FIG. 1, the reference numbers are also kept the same, but with a “2” as the initial digit. Thus, the nappy processing apparatus 200 includes an opening 205 through which at least one nappy is received into a chamber 210. A heating member 230 is also provided, which is adapted to provide heat to a nappy in the chamber 210. As will be understood, while the apparatus 200 is described as a nappy processing apparatus, it would also be suitable for processing alternative domestic waste, such as waste foodstuffs.

The apparatus 200 further includes a blade member 215 arranged to project into the chamber 210. The blade member 215 is actuatable so as to agitate the at least one nappy in the chamber.

The blade member 215 projects into the chamber 210 from the wall 214. In the example shown, the blade member 215 projects from radially the wall 214 towards the central axis of the chamber 210.

The blade member 215 projects above the heating member 230. That is the axial distance from the base 212 to the blade member 215 is greater than the axial distance from the base 212 to the distal end of the heating member 230.

The blade member 215 is an elongated blade member which extends around the wall 214, projecting into the chamber 210. In particular, the blade member 215 extends around a portion of the circumference of the wall. In this example, the portion is an arc of around 20° of the circumference. The elongated blade member 215 extends parallel to the base 212.

The chamber 210 is rotatably mounted in the apparatus 200. In the example shown, the wall 214, base 212, and blade member 215 are integrally formed so as to be rotated together by the rotator 250. The heating member 230 projects into the chamber 210 but is not integrally formed with the base 212. In this way the chamber 210 may be rotated relative to the heating member 230. The blade member 215 may be rotated relative to the heating member 230. The heating member 230 is static.

The blade member 215 is actuated by rotation of the chamber 210. Rotation of the blade member 215 agitates the nappy or nappies held within the chamber 210. In the example shown, agitation enables the blade member 215 to provide a grinding action to the nappy or nappies in the chamber 210. In particular, the blade member 215 grinds the nappy or nappies in the narrowed space between the blade member 215 and the heating member 230.

An electric motor 270 is provided to actuate the rotator 250 and thereby rotate the chamber 210. The electric motor 270 is also connected to a controller to enable the electric motor to be selectively activated and thereby selectively rotate the chamber 210. Consequently, the controller enables the apparatus to selectively agitate the nappy or nappies held in the chamber 210. The controller may be configured to determine the rotational speed and the time period for which the chamber 210 is rotated. The controller may thereby be configured to determine the rotational speed and the time period for which the nappy or nappies are agitated.

The heating member 230 includes an electrical heating element, in a similar manner as the example of FIG. 1. The controller selectively activates the heating element 230 as is described herein. The controller thereby coordinates both activation of the heating element 230 as well as agitation provided by the blade member 215.

It will be appreciated that the heating member of each of the examples described herein may be configured to provide heat to the nappies in a number of ways. For example, the heating member may provide heat independently of, or in coordination with, other features of the apparatus, such as the actuation of one or more blade members, or the rotation of the chamber.

In this way, the controller may be configured to determine the time period that the heating member provides heat to the nappies in the chamber. Additionally, or alternatively, the controller may be configured to determine the temperature to which the nappies in the chamber are heated. The controller may be configured to enable the heating member to provide continuous heat to the nappies in the chamber or intermittent heat to the nappies in the chamber.

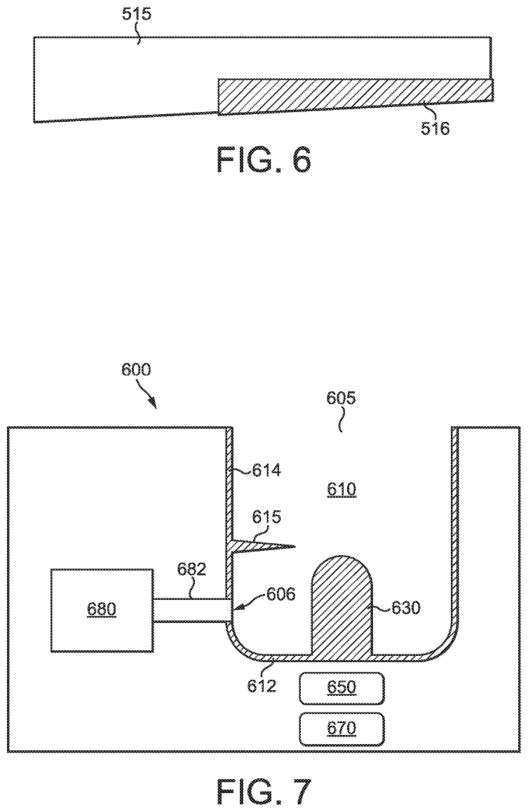

The controller may be configured to vary both the heating and rotation. In alternative arrangements, heating and rotation may be each be varied by independent controllers. Referring now to FIG. 6, there is shown an illustration of a blade member 515. The blade member 515 is mounted to a wall of a chamber in the same manner as the blade member described above with reference to FIG. 2.

The blade member 515 includes a contacting element 516. The contacting element 516 is adapted to at least partially disintegrate the nappy or nappies deposited in the chamber as they are agitated by the blade member.

In the example shown, the contacting element 516 includes a roughened surface on the underside of the blade member 515. As the blade member 515 agitates the nappy, the contacting element 516 pulls at the surface layers of the nappy to initiate disintegration of the nappy during processing. Further processing may cause the contacting element to abrade or cut through fibres of the nappy, accelerating disintegration. As the nappy is disintegrated, the associated heat dehydrates the nappy more quickly and evenly. Optionally, the contacting element 516 would also be suitable for agitating and causing disintegration of alternative domestic waste, such as waste foodstuffs.

As will be appreciated, the contacting element may be provided in a number of suitable forms and arrangements that at least partially disintegrates a nappy during processing. For example, the contacting element may include a plurality of projections or serrations, or may include one or more patterned or textured regions. The plurality of projections, plurality of serrations, patterned region or textured region may be formed from either a regular, or an irregular, arrangement of a number of individual components.

Suitably, the one or more contacting elements may be provided on any surface of the apparatus which is contactable with the nappy during processing. For example, a contacting element may be provided on any exposed surface of a blade member or a wall of the chamber into which the blade member projects. The contacting element may also be provided on a heating member of the apparatus, or on its removable lid. Referring additionally to FIG. 4, there is shown a flow chart of a method 400 of processing waste, such as a nappy. The method may be carried out using an example apparatus such as example apparatus 200 described with reference to FIG. 2.

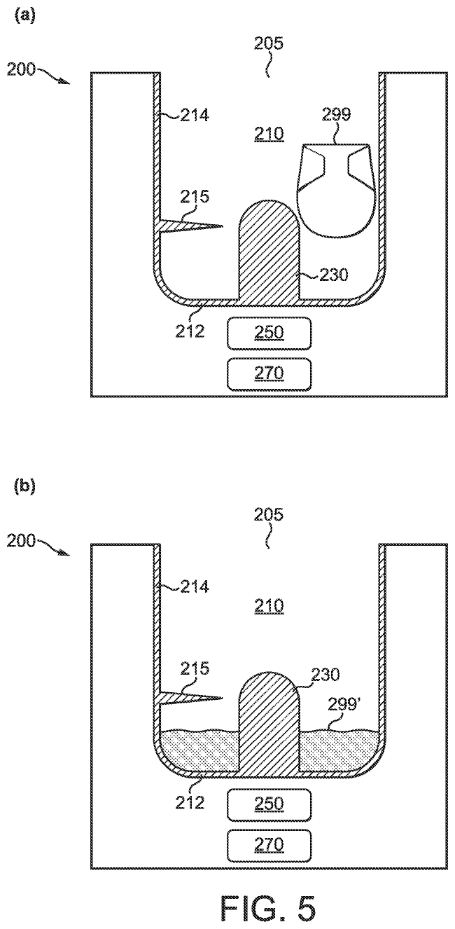

The method of FIG. 4 includes a first step 410 corresponding to the method described with reference to FIG. 3. Thus, the first step 410 includes depositing at least one nappy into a chamber through a first opening of the chamber. Suitably, as shown in FIG. 5a), the at least one nappy 299 may be a used nappy, containing liquid and solid waste, formed into a rough ball and secured using the nappy fastening tapes.

The second step 420 includes providing heat to the at least one nappy within the chamber. Suitably, the heat is provided by a heating member. The heating member may include, for example, a heating element and controller as described with reference to FIG. 3, to selectively provide heat and control cycle length.

A third step 430 includes agitating the at least one nappy within the chamber by actuating a blade member arranged to project into the chamber. Suitably, actuation of the blade member enables the blade member to provide a grinding action to the nappy or nappies in the chamber.

Additionally, or alternatively, actuation of the blade member may include partially disintegrating the at least one nappy with a contacting element, such as the contacting element as described herein.

Processing a waste using the above method uses a combination of heating and agitating to dry and disintegrate the waste. For example if the waste is a nappy 299, then over a period of several hours, typically from 4 hours to 10 hours, the nappy 299 is broken down into fragments 299′, as shown in FIG. 5b). The fragments 299′ ensure that the nappy is in a form that will more readily decompose. Furthermore, processing multiple nappies to provide dried fragments ensure they occupy a significantly reduced volume both in a user's temporary storage and in landfill.

Referring now to FIG. 7, there is shown a further nappy processing apparatus 600. Where the features are the same as the example of FIG. 2, the reference numbers are also the same, but with a “6” as the initial digit. Thus, the nappy processing apparatus 600 includes an opening 605 through which at least one nappy is received into a chamber 610. A heating member 630 is also provided, which is adapted to provide heat to a nappy in the chamber 610. The apparatus 600 further includes a blade member 615 arranged to project into the chamber 610, wherein the blade member 615 is actuatable so as to agitate the at least one nappy in the chamber. Again, while the apparatus 600 is described as a nappy processing apparatus, it would also be suitable for processing alternative domestic waste, such as waste foodstuffs.

The apparatus further includes a conduit 682 for extracting air from the chamber 610. The conduit is fluidly connected to the chamber 610 by a second opening 606 in the chamber 610. The second opening 606 is provided in the wall 614 of the chamber 610. Alternatively, the second opening may be provided in the base, the heating member or the blade member.

In the example shown, the conduit 682 is fluidly connected to a fan unit 680. The fan unit 680 is adapted to extract air from the chamber 610 via the conduit during use. In this way, a negative pressure relative to the ambient atmosphere is maintained within the chamber 610.

The fan unit 680 also includes a filter element (not shown). The filter element is configured to filter the air extracted from the chamber 610 by the fan unit 680. Suitably, the filter element 680 is configured to deodorise, or to trap or collect odours from, the air passing through. In an example, the filter element includes activated carbon.

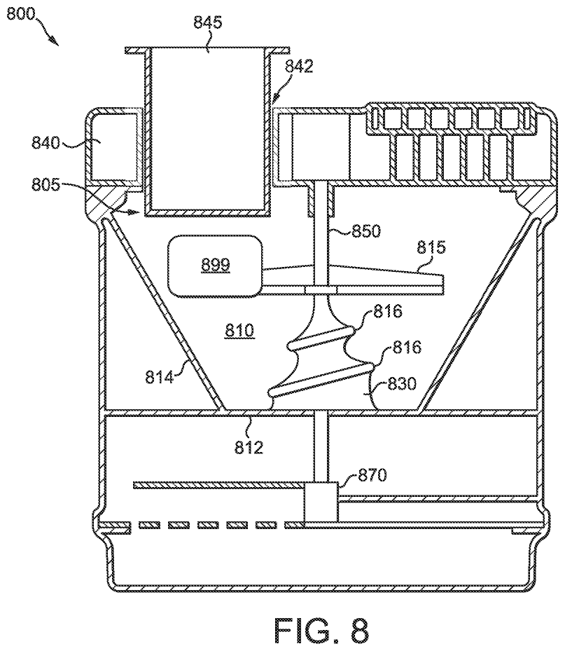

Referring now to FIG. 8, there is shown a waste processing apparatus 800 with a heating member 830 including a contacting element 816 thereon. Where the features are the same as the example of FIG. 1, the reference numbers are also kept the same, but with a “8” as the initial digit. Thus, the waste processing apparatus 800 having a chamber 810 including a heating member 830 adapted to provide heat to a portion of waste 899 in the chamber 810. The waste received into the chamber may be nappy waste and/or waste foodstuff.

The chamber 810 includes an opening 805 through which the waste is received into a chamber 810. A lid 840 is fitted within the opening 805. The lid 840 closes the opening 805 to provide an air tight seal to the first opening 805 during use. In this way, odours emit from the portion of waste 899 during use are retained with the chamber 810 and isolated from the surrounding environment.

The lid 840 includes an inlet 842 through which the waste 899 is received into the first opening 805 of the chamber 810. The inlet 842 has a moveable closure means 845, arranged therein. In the example, the moveable closure means 845 is plunger that is selectively removeable from the inlet 842. With the plunger removed from the inlet 842, the user is able to deposit waste 899 into the chamber 810. With the waste 899 deposited, the user inserts the plunger into the inlet 842 for use, sealing the inlet 842.

The heating member 830 extends into the chamber 810 from the base 812 along the longitudinal central axis of the chamber 810. A proximal end of the heating member 830 is provided on the base 812. A distal end of the heating member 830 is disposed away from the base 812. The proximal portion is wider than the distal portion so that the heating member is a substantially conical shape. The heating member 830 thereby has an increased surface area available to provide heat to the waste to efficiently and effectively heat waste received in the chamber 810.

The heating element 830 is mounted to a spindle 850 for rotation by an electric motor 870. The spindle 850 is oriented along the central axis of the chamber. The spindle 850 is operably mounted to an electric motor 870 to rotate the heating element 830 relative to the chamber 810. The heating member 830 includes an electrical heating element connected to a suitable electrical circuit, including a controller (not shown), to enable the heating element 830 to be selectively activated.

The heating element 830 includes a contacting element 816 mounted on the heating member 830 to project into the chamber 810. The contacting element 816 is an elongate helical rib extending around an outer surface of the heating member 830. The helical rib extends around the heating member 830 from the proximal portion to the distal portion of the heating member 830. In this way, the contacting element 816 is akin to a screw thread on the outer surface of the heating element 830, configured to use the thread to disintegrate the waste 899 into fragments.

The side wall 814 of the chamber is tapered so as to be narrower towards the base 812 than distal to the base 812. The lateral space between the side wall 814 and the contacting element 816 decreases towards the base 812 of the chamber 810. Thus, in use, as the waste 899 is broken into smaller fragments, the fragments accumulate towards the base 812 of the chamber 810 maintaining contact with the contacting element 816 for effective agitation.

The apparatus 800 further includes a blade member 815. The blade member 815 is mounted to the spindle 850 for co-rotation with the heating member 830. By mounting both the blade member 815 and the heating member 830 to the same spindle 850 the rotation may be driven by a single electric motor. The blade member 815 is actuatable so as to agitate the waste, at least initially, within the chamber 810 directing the waste 899 towards the base 812 and onto the contacting element 816.

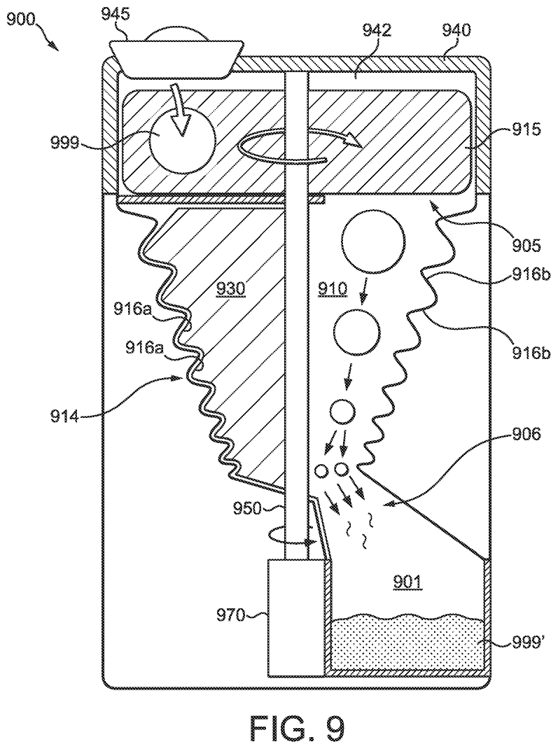

Referring now to FIG. 9, there is shown another waste processing apparatus 900 with a heating member 930 including a contacting element 916 thereon. Where the features are the same as other examples, the reference numbers are also kept the same, but with “9” as the initial digit. Thus, the waste processing apparatus 900 has a chamber 910 including a heating member 930 adapted to provide heat to a portion of waste 999 in the chamber 910. The waste received into the chamber may be nappy waste and/or waste foodstuff.

The apparatus 900 includes a lid 940 having an inlet 942 through which the waste 999 is conveyed into the first opening 905 of the chamber 910. The inlet 942 includes a removable cover 945 for sealing an entrance to the inlet 942. With the cover 945 removed from the entrance, the user is able to deposit a waste into the inlet 942.

The apparatus 900 includes a blade member 915 rotatably mounted on a spindle 950 and positioned within the inlet 942. The spindle 950 is oriented vertically within the apparatus 900 so that the blade member 942 rotates horizontally about the spindle 950 within the inlet 942.

The blade member 942 includes a series of individual blades, typically four, extending from the spindle 950 to a periphery of the inlet 942. After depositing waste into the inlet 942, the blade member 942 is rotated by the driving the spindle 950 to convey the waste 999 to the first opening 905. In this way, the series of blades form an air lock system between the entrance to the inlet 942 and the chamber 910, so that a portion of waste 999 can be deposited within the chamber without odour venting back through the entrance of the lid 940.

The chamber 910 includes a first opening 905 for receiving waste 999, as well as a second opening 906 configured to discharge processed waste 999′ from the chamber 910. Waste deposited into the first opening 905 from the inlet 942 is processed as it passes through the chamber 910 to the second opening 906. Thus, the apparatus 900 is configured for continuous processing of waste 999.

A heating element 930 is mounted to the spindle 950 to rotate with the spindle 950. The heating element 930 and blade member 915 are co-mounted to the spindle 950 so that each can be driven by a single electric motor 970.

The heating element 930 extends radially from the spindle 950. The heating element extends from proximal to the base 912 to the opening 905 at the top of the chamber 910. In this way, the heating element 930 is configured as a blade member 915, that is a second blade member, within the chamber 910.

In an alternative arrangement, a heating element may be provided within the side wall and/or within the base of the chamber. In this way, the waste is heated by contact with the side wall and/or wall of the chamber In this arrangement, the second blade member is used primarily to agitate and disintegrate the waste into fragments.

The side wall 914 of the chamber 910 is tapered. The side wall 914 has a smaller lateral width at the base 912 compared with the lateral width distal to the base 912. The heating element 930 is also tapered such that the radius towards the base 912 of chamber 910 is less than the radius at the top of the chamber 910.

The side wall 912 has a first contacting element 916a extending around its side wall 914. The first contacting element 916a forms a series of annular projections in the side wall 914.

The heating element 930 has a second contacting element 916b extending along its peripheral edge. The second contacting element 916b is a series of serrations on the peripheral edge.

The first contacting element 916a is cooperatingly engaged with the second contacting element 916b. In particular, the dimensions and spacings of the series of serrations are configured to be alternately located between the annular projections of the side wall 914. Furthermore, the lateral space between the first contacting element 916a decreases towards the base 912 of the chamber 910. The angle of taper of the side wall 914 is further from the vertical than the angle of taper of the outer surface of the heating member 930. Thus, in use, as the waste 999 is broken into smaller fragments, the fragments are processed closer to the base 912 of the chamber 930. The fragments of processed waste 999′ are directed towards the second opening 906 by cooperative action between the tapered side wall 914 and tapered heating member 930.

The apparatus 900 includes a storage chamber 901. The second opening 906 opens into storage chamber 901 so that processed waste 999′ is discharged from the chamber 910 into the storage chamber 901. Typically a removable receptacle is provided in the storage chamber 901 to enable easy removal of the processed waste 999′.

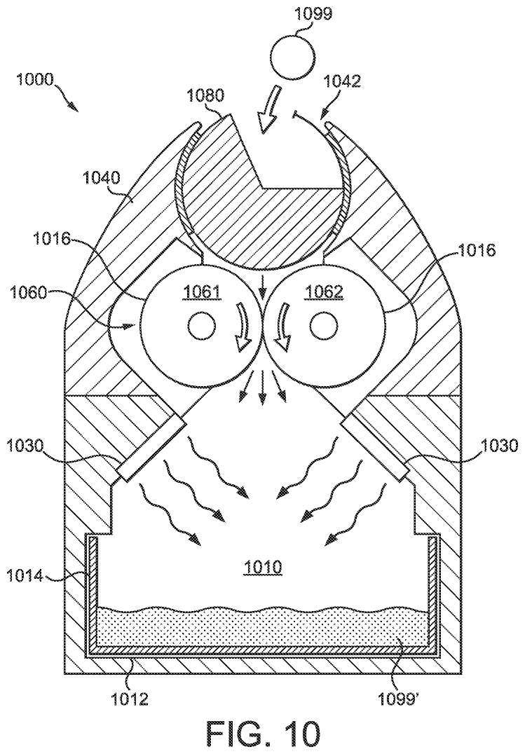

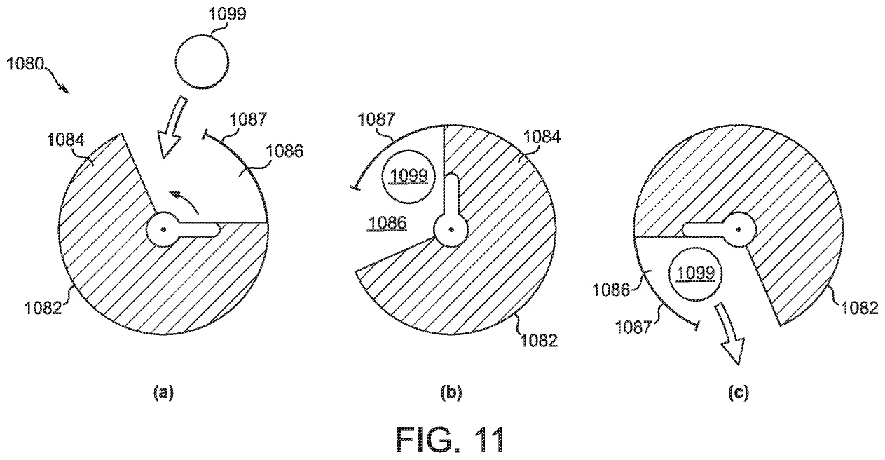

Referring now to FIGS. 10 and 11, there is shown a further waste processing apparatus 1000. Where the features are the same as other examples, the reference numbers are also kept the same, but with “10” as the initial digits. Thus, the waste processing apparatus 1000 has a chamber 1010 including a heating member 1030 adapted to provide heat to a portion of waste 1099 in the chamber 1010. The waste received into the chamber may be nappy waste and/or waste foodstuff.

The apparatus 1000 includes a lid 1040 having an inlet 1042 through which the waste 1099 is conveyed to the first opening 1005 of the chamber 1010. The inlet 1042 includes a moveable closure means 1080, as shown in detail in FIG. 11. The closure means 1080 receives a portion of waste 1099 into the inlet 1042 and is actuated to rotates a roller 1084 to capture the waste 1099 and pulls it through the inlet 1042.

Referring particularly to FIG. 11, there is shown a moveable closure means 1080 having a roller 1084 arranged in the inlet 1042 with a horizontal rotational axis. The roller 1084 includes an outer surface 1082 with a recessed channel 1086 in the outer surface 1082. The recessed channel 1086 extends along the outer surface 1082 in an axial direction. The recessed channel 1086 is partially covered by an annular wall portion 1087.

In use, the roller 1084 is presented in a starting position, as shown in FIG. 11 a) in which the recessed channel 1086 is oriented upwards. The recessed channel 1086 is oriented towards the entrance of the lid 1040 to receive a portion of waste 1099.

After the waste 1099 is deposited in the recessed channel 1084, the roller 1084 is rotated around its axis, moving the waste 1099 through the inlet 1042, as shown in FIG. 11 b).

With further rotation of the roller 1084, the recessed channel 1084 is oriented towards the opening 1005 and the waste 1099 passes out of the inlet 1042, typically under gravity, for subsequent processing within the apparatus 1000.

The apparatus 1000 further includes a shredding assembly 1060. The shredding assembly 1060 is mounted in the underside of the lid 1040 to enable removal from the main housing of the apparatus 1000. Alternatively, a shredding assembly may be separately mounted to the housing of the chamber, for example by being positioned over the opening and locked between the housing and the lid by mounting the lid to the housing.

The shredding assembly 1060 has a pair of rotating barrels, 1061, 1062, each including a respective contacting element 1016 on its outer surface. In the example, each contacting element 1016 is a plurality of serrations provided on the outer surface of the rotating barrel 1061, 1062. The serrations of each barrel 1061, 1062 are interlaced with one another during rotation.

The pair of rotating barrels 1061, 1062 are arranged with parallel rotational axes and coordinated to rotate counter to one another. In this way, when the user actuates the shredding assembly, both barrels 1061, 1062 rotate in opposing angular directions so that the respective serrations of each contacting element 1016 cooperatingly capture and disintegrate the waste 1099 as it passes between the barrels 1061, 1062 and into the receiving chamber 1030.

The apparatus 1000 further has a heating member 1030 in the chamber 1010, in the form of a pair of heat lamps mounted within the chamber 1010. Typically, the heat lamps are halogen lamps are positioned either side of the opening 1005 and angled to irradiate the disintegrated waste 1099′held in a receptable at the base 1012 of the chamber 1010. Typically the receptacle is a removable receptacle provided, enabling easy removal of the processed waste 1099′from the chamber 1030.

By arranging a shredding assembly upstream of the chamber 1030, the waste 1099 may be substantially disintegrated prior to heating. Disintegrating the waste prior to entering the chamber 1030, the speed with which waste 1099 is processed is increased.

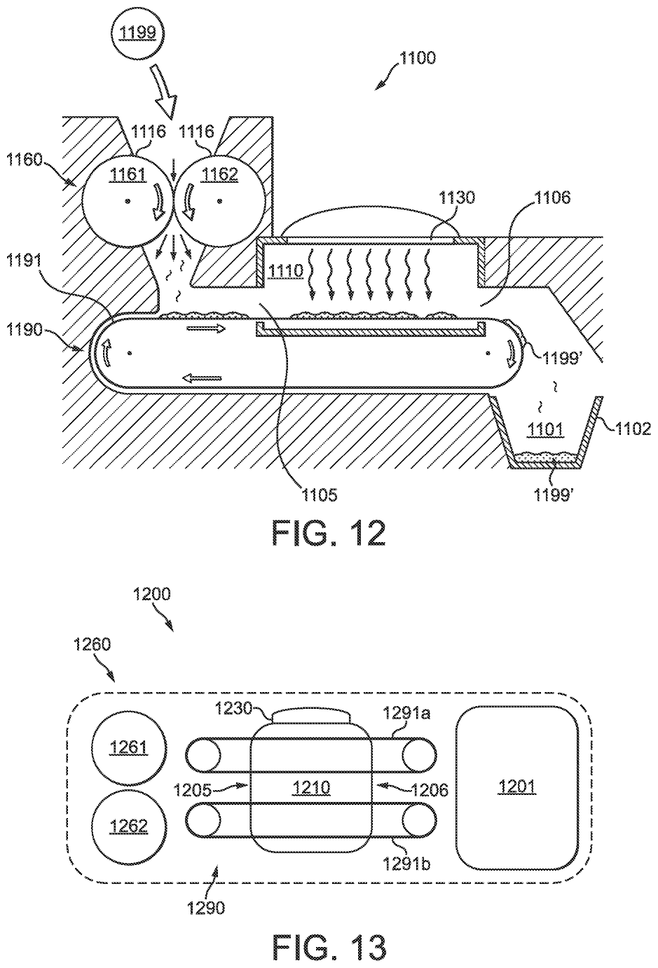

Referring now to FIG. 12, there is shown a further example waste processing apparatus 1100. Where the features are the same as other examples, the reference numbers are also kept the same, but with “11” as the initial digits. Thus, the waste processing apparatus 1100 has a chamber 1110 including a heating member 1130 adapted to provide heat to a portion of waste 1199 in the chamber 1110. The waste received into the chamber may be nappy waste and/or waste foodstuff.

The apparatus 1100 has chamber 1110 including a first opening 1105 for receiving waste, as well as a second opening 1106 configured to discharge the processed waste 1199′ from the chamber. Waste 1199 is deposited into the first opening 1105 from a shredding assembly 1160 substantially the same as the shredding assembly described with reference to FIGS. 10 and 11. The shredding assembly is located within an inlet 1142.

The apparatus 1100 includes a conveyor assembly 1190 to convey waste from the first opening 1105 to the second opening 1106. The conveyor assembly 1190 includes a moveable belt 1191 extending beyond the chamber 1130, from upstream of the first opening 1105 to downstream of the second opening 1106.

The conveyor assembly 1191 includes a pair of pulleys (not shown) with the moveable belt secured around each pulley. At least one of the pulleys is motorised to drive the moveable belt in use.

The chamber 1110 includes a heating member 1130, typically a heat lamp mounted within the chamber 1110. The heating member 1130 is positioned above the moveable belt 1191 of the conveyor assembly 1190, and oriented to irradiate the disintegrated waste 1199′ as it is conveyed through the chamber 1110.

In use, the conveyor assembly 1190 is operably connected to a controller (not shown). Thus controller is configured to adjust a speed of the conveyor assembly to determine the duration that the waste 1199 is within the chamber during processing. Additionally, the controller is also connected to the heating member 1130. The controller is also configured to adjust the heating of the waste 1199 in the chamber 1110.

The apparatus 1100 also includes a storage chamber 1101. The second opening 1106 opens into storage chamber 1101. The conveyor assembly 1190 extends into the storage chamber 1101 and is oriented so that processed waste 1199′ is discharged from the belt 1191 into the storage chamber 1101. Typically a removable receptacle 1102 is provided in the storage chamber 1101 to enable easy removal of the processed waste 1199′.

Referring now to FIG. 13, there is shown a further example waste processing apparatus 1200. Where the features are the same as other examples, the reference numbers are also kept the same, but with “12” as the initial digits. The waste processing apparatus 1200 is substantially the same as the apparatus 1100 described with reference to FIG. 12 other than the conveyor assembly 1290 includes a pair of moveable belts oriented to cooperatingly engage the waste and convey the waste through the chamber. In the example, a first belt 1291a of the pair is oriented to move in parallel to a second belt 1291b. The conveyor assembly 1290 is operably connected to a controller for use in the same manner as the other examples described herein.

Various modifications to the apparatus described herein are possible.

In certain examples, the blade member may be provided to project from any suitable surface so as to project within the chamber.

Multiple blade members may be provided, either on the same surface or different surfaces. For example, a first blade member may be provided on the base of the chamber and a second blade member may be provided on the wall of the chamber, or on the heating member. Each blade member may have a different orientation and shape. Each blade member may have different or matching contacting elements.

One or each blade member may be an elongated blade member. An elongated blade member may extend circumferentially, axially, radially or any combination thereof. An elongated blade may extend parallel to, orthogonal to or at an acute angle to a base or wall of the chamber. In an example, the blade member may extend along the base.

In these ways, the blade members may be modified so as to optimise agitation of the waste in the chamber. Each blade member may be modified to optimise disintegration of the waste that are being processed, such as by including one or more contacting elements.

In apparatus with at least two blade members, the blade members may each be agitated in the same manner, or differently. For example, a first blade member may be actuated to rotate in a first direction around the central axis of the chamber, and a second blade member may be actuated to rotate is second, opposing direction. Alternatively, a first blade member may be actuated and a second blade member may be static.

In certain examples, blade members are actuated to rotate within the apparatus. Additionally, or alternatively a blade member may be actuated by other movement or combinations of more than one movement. For example, blade member may project into the chamber along a blade axis. The blade member may be actuated by pivoting or rotating around the blade axis. The blade member may be actuated by extending or withdrawing along the blade axis.

In certain examples, waste may be agitated within the chamber by means other than a blade member. For example, waste may be agitated by a jet of air, such as provided by a fan unit and conduit directing air into the chamber through a second opening. Thus, the conduit and fan unit as described above with reference to FIG. 7 may be reconfigured to provide air to the chamber via the second opening. The air may be directed into the chamber in pulses.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A waste processing apparatus comprising:
a chamber having a first opening through which a portion of waste is received into the chamber; and
a heating member adapted to provide heat to the portion of waste in the chamber.

2. A waste processing apparatus according to claim 1, wherein the chamber is rotatable.

3. A waste processing apparatus according to claim 1, wherein the chamber comprises a base and a side wall extending away from the base.

4. A waste processing apparatus according to claim 1, wherein the base wall and/or the side wall is metallic.

5. A waste processing apparatus according to claim 1, wherein the chamber is configured to form a passageway and the portion of waste passes through the passageway as the heating member provides heat.

6. A waste processing apparatus according to claim 1, further comprising a second opening configured to discharge the portion of waste from the chamber.

7. A waste processing apparatus according to claim 6, further comprising a conveyor assembly to convey the portion of waste from the first opening to the second opening.

8. A waste processing apparatus according to claim 7, wherein the conveyor assembly comprises a pair of moveable belts oriented to cooperatingly engage the portion of waste and convey the portion of waste through the chamber.

9. A waste processing apparatus according to claim 1, further comprising a lid for selectively closing the first opening.

10. A waste processing apparatus according to claim 1, wherein the lid comprises a inlet having a moveable closure means configured to selectively seal the inlet.

11. A waste processing apparatus according to claim 1, wherein the apparatus further comprises at least one blade member arranged to project into the chamber, wherein the at least one blade member is actuatable so as to agitate the portion of waste in the chamber.

12. A waste processing apparatus according to claim 1, further comprising at least one contacting element, wherein each contacting element is adapted to at least partially disintegrate the portion of waste.

13. A waste processing apparatus according to claim 12, further comprising a shredding assembly, wherein the shredding assembly has at least one contacting element moveably mounted therein.

14. A waste processing apparatus according to claim 13, wherein the shredding assembly is positioned at an entrance to the first opening.

15. A waste processing apparatus according to claim 12, wherein the at least one contacting element is mounted on a blade member.

16. A waste processing apparatus according to claim 1, wherein the heating member is adapted to provide heat for a predetermined period of time.

17. A waste processing apparatus according to claim 1, wherein the heating member is adapted to provide heat intermittently.

18. A waste processing apparatus according to claim 1, wherein the heating member provides heat to increase a temperature of the portion of waste to at least 50 C., preferably at least 70 C.

19. A waste processing apparatus according to claim 1, wherein the heating member is arranged so that at least a portion of the heating member projects into the chamber.

20. A waste processing apparatus according to claim 1, further comprising a conduit for extracting air from chamber so as to maintain negative pressure within the chamber during use, and optionally, wherein the conduit is fluidly connected to a filter element.

21.-25. (canceled)
